(19) United States
(12) Patent Application Publication
(10) Pub. No.: US 2024/0180942 A1
(43) Pub. Date: Jun. 6, 2024

(54) NUTRITIONAL SUPPLEMENT AND USES

(71) Applicant: Citrox Biosciences Limited, Kimbolton, Cambridgeshire (GB)

(72) Inventor: Richard Thomas, Kimbolton, Cambridgeshire (GB)

(73) Assignee: Citrox Biosciences Limited, Kimbolton, Cambridgeshire (GB)

(21) Appl. No.: 18/287,530

(22) PCT Filed: Apr. 19, 2022
(86) PCT No.: PCT/GB2022/050975

§ 371 Date: Oct. 19, 2023

(30) Foreign Application Priority Data
Apr. 19, 2021 (GB) 2105574.4

(51) Int. Cl. A61K 31/7048; A61K 9/00; A61K 9/20; A61K 9/48; A61K 47/26; A61K 47/38; A61P 1/04; A61P 3/06; A61P 3/10

(52) U.S. Cl. CPC A61K 31/7048; A61K 9/0095; A61K 9/2009; A61K 9/2013; A61K 9/4825; A61K 47/26; A61K 47/38; A61P 1/04; A61P 3/06; A61P 3/10

(57) ABSTRACT

The present invention relates to a nutritional supplement for use by oral administration to a human for ameliorating adverse medical conditions deriving at least in part from a metabolic dysfunction and/or an intestinal dysfunction, which supplement is in a form suitable for oral administration and comprises a mixture of bioflavonoids which include at least 45% wt/wt naringin and at least 15% wt/wt of neohesperidin of the bioflavonoids present. The present invention also provides a method of treatment of the aforementioned medical conditions using the aforementioned nutritional supplements.

### CROSS-REFERENCE

This application is a 371 National Stage filing and claims the benefit under 35 U.S.C. § 120 of International Application No. PCT/GB2022/050975, filed 19 Apr. 2022, which claims priority to Great Britain Application No. GB2105574.4, filed Apr. 19, 2021, each of which is incorporated herein by reference in its entirety.

### AREA OF THE INVENTION

The present invention relates to a nutritional supplement which can help ameliorate various medical problems which result at least in part from gastrointestinal and/or metabolic conditions. More particularly, the invention comprises naringin and neohesperidin, which are bioflavonoids derivable from oranges, especially from the pith of immature bitter oranges, for use to ameliorate such conditions as type 2 diabetes or prediabetes, gastro-intestinal ulcers such as duodenal ulcers, disadvantageous gut permeability, inflammatory and other immune conditions and dysbiosis. The nutritional supplement is aptly in a form administrable by mouth, for example as a solid unit dosage form, a liquid unit dosage form or a liquid multidosage form from which a unit dosage may be withdrawn. The present invention also provides a method of treatment of the aforementioned medical conditions using the aforementioned nutritional supplements.

### BACKGROUND TO THE INVENTION

Natural products have been suggested as suitable agents for use to improve intestinal function. Most commonly, these natural products are derived from grapefruit and are commonly used as a purified single compound. Possibly, the most commonly referred to agent of this type is quercetin. However, as yet there appears to be insufficient commonly available data to conclude that quercetin (or any other bioflavonoid) has been demonstrated to be effective in humans for the treatment of conditions such as leaky gut syndrome, irritable bowel syndrome, or improvement in microbiota which can assist in enhancing bodily defenses against disease and metabolic disfunctions. Possibly, this has been because of concerns about the variable bioavailability of various bioflavonoids.

Many references occur in the literature to use of various individual flavonoids in various animals for treatment of various conditions. However, no references occur in the literature in respect of orally administering naturally occurring mixtures of flavonoids to humans that result in demonstrated amelioration of adverse medical conditions.

The number of naturally occurring flavonoids is extremely large, and many sources of these agents are known. The number of naturally occurring phytochemicals is enormous, and the food sources for such phytochemicals are similarly extensive.

Bioflavonoids derivable from the pith of the immature bitter orange are disclosed in, for example, WO 2008/009956, WO 2008/09958, WO 2010/089600, WO 2012/017186 and WO 2014/030005.

Such compositions have been disclosed primarily because of antibacterial properties which permits, for example, their use in compositions such as toothpastes and mouthwashes. Particular flavoured compositions of this type have been commercially available under the name Citrox™. Such products have been labelled as not for internal consumption.

However, it has now been discovered that oral ingestion of naringin and neohesperidin as a nutritional supplement have beneficial effects on various medical conditions in humans. Thus, for example, daily oral ingestion of these bioflavonoids has been found to aid in healing of duodenal ulcers, reduce blood sugar levels in type 2 diabetes and reduce hyperpermeability of the gut, which in turn will be of aid in ameliorating adverse conditions associated therewith and aid in reducing adiposity, such as central adiposity, in overweight individuals.

The nutritional supplement may be considered to be a pharmaceutical composition comprising a mixture of bioflavonoids and a pharmaceutically acceptable carrier therefor.

### SUMMARY OF THE INVENTION

The present invention provides a nutritional supplement for use by oral administration in ameliorating adverse gastrointestinal or metabolic medical problems in a human, which nutritional supplement is in a form suitable for oral administration and comprises a mixture of bioflavonoids which include at least naringin and neohesperidin, wherein the naringin comprises at least 45% wt/wt of the bioflavonoids and neohesperidin comprises at least 15% wt/wt of the bioflavonoids.

More aptly, the naringin comprises at least 50% wt/wt of the mixture of bioflavonoids. More aptly, the neohesperidin comprises at least 20% wt/wt of the mixture of bioflavonoids.

Preferably, naringin and neohesperidin together comprise at least 75% wt/wt of the mixture of bioflavonoids, for example 80-90% of the mixture of bioflavonoids.

The nutritional supplement may be provided in a form suitable for oral administration so that the bioflavonoids are made available within the gastrointestinal tract, wherein absorption results in systemic availability of the bioflavonoids.

It has now been found that nutritional supplements derived from oranges, and in particular the immature bitter oranges, produce desirable effects after oral administration. Aptly, such supplements also comprise biomass derived from immature bitter oranges, such as from the pith of the immature bitter orange. Such biomass aids in solubilisation of the bioflavonoids (and hence bioavailability). Such solubilizing biomass is frequently polysaccharide in nature.

Such nutritional supplements will comprise naringin and neohesperidin, and optimally one or more of the other flavonoids shown in Reference Example 1 herein, optionally together with biomass, as referred to in Reference Example 2 herein.

Such nutritional supplements may also comprise one or more carboxylic acids, such as citric acid, tartaric acid, lactic acid and caprylic acid. Aptly, such nutritional supplements will comprise citric acid, tartaric acid and lactic acid, as set out in Reference Example 3 herein.

The nutritional supplement may comprise a solid unit dosage form, of which capsules and tablets are particularly apt, or other forms such as sachets containing powders or granules may be employed.

The nutritional supplement may also be provided in liquid form, where it may be provided, for example, in unit dosage form, for example a sachet, or in a liquid multiple dosage form such as a bottle from which a measured unit dosage form may be obtained; for example, a 5 ml spoon, or a bottle cap of defined volume, or the like may be used to provide an appropriate unit dose from a bottle.

Tablets may be readily dispensable to provide a drinkable solution or dispersion if desired, for example effervescent tablets may be used to provide a drinkable unit dose solution.

Swallowable capsule forms are particularly favoured. Such swallowable capsules may comprise a hard shell, for example of gelatine, filled with a unit dose, for example as set out in Reference Examples 2 and 3.

The unit dose of the nutritional supplement may provide from 50 mg to 2500 mg of bioflavonoids as set out hereinbefore; more aptly, for example, from 100 mg to 2000 mg of bioflavonoids, and favourably from 200 mg to 1000 mg of bioflavonoids, for example 250 mg or 500 mg of a mixture of bioflavonoids as set out hereinbefore.

Such dosage forms may be taken as desired, for example once every other day to eight or more times per day, but most aptly are administered from 1 to 6 times a day; for example, 1, 2, 3 or 4 times a day.

Typically, the daily dose of bioflavonoids will be from 100 mg to 2500 mg; for example, 200 mg to 2000 mg, such as 200, 250, 300, 350, 400, 450, 500, 600, 700, 750, 800, 1000, 1250 or 1500 mg.

Advantageously, the orally administrable nutritional supplement will also comprise residual biomass derived from oranges, for example as in Reference Example 2 herein.

The nutritional supplement thus administered can help ameliorate numerous adverse medical conditions, for example those which may at least in part be caused by adverse effects in the gastrointestinal tract.

The nutritional supplement may be administered prophylactically in order to prevent an anticipated adverse medical condition, or may be administered after the condition is manifest. In the latter case, the nutritional supplement may be administered alone, but may be administered together with a pharmaceutical composition for treating the medical condition.

Thus, for example, if a person suffers from type 2 diabetes, the nutritional supplement herein may be administered alone or as adjunct to a pharmaceutical therapy such as metformin treatment. On the other hand, if a person is deemed pre-diabetic and not yet requiring pharmaceutical intervention, then the nutritional supplement herein may be administered, to reduce the likelihood of progression to type 2 diabetes. In both such cases, the person may be advised to reduce calorie intake and/or increase exercise, as well as employ the nutritional supplement herein.

Even without calorie restriction and/or increased exercise, the use of the nutritional supplement herein can lead to a reduction in excess adiposity, which can also be beneficial to the patient and may manifest as weight loss or a reduction in waist size. However, calorie restriction and/or exercise is likely to enhance these desirable effects. Reduction in central adiposity can also assist in ameliorating non-alcoholic fatty liver disease.

Similarly, if a human is suffering from a gastrointestinal ulcer, the patient may be treated with an agent such as omeprazole, ranitidine and/or amoxicillin or the like, and may also take the nutritional supplement herein to further aid in reduction of the problem. However, use of the nutritional supplement herein alone can benefit the sufferer and so reduce the reliance on pharmaceutical intervention.

Other conditions which may be addressed by use of the nutritional supplements herein include those associates with the so-called metabolic syndrome. While not wishing to be bound by any theory, the benefits may derive from an avoidance of insulin spiking, which becomes reduced by use of the nutritional supplement.

### DETAILED DESCRIPTION OF THE INVENTION

The present invention provides a nutritional supplement for use by oral administration in a human ameliorating adverse medical conditions deriving at least in part from metabolic and/or intestinal dysfunction, which supplement is in a form suitable for oral administration and comprises a mixture of bioflavonoids which include at least 45% wt/wt of naringin and at least 15% wt/wt of neohesperidin of the bioflavonoids present.

From an alternative viewpoint, the present invention provides a method of ameliorating an adverse medical condition in a human which derives at least in part from a metabolic and/or intestinal dysfunction which method comprises the oral administration of an effective amount of a nutritional supplement which comprises a mixture of bioflavonoids which include at least 45% wt/wt of naringin and at least 15% wt/wt of neohesperidin of the bioflavonoids present.

A nutritional supplement may be considered to be a composition comprising active materials that can be derived from a natural diet which includes plant-derived products, especially citrus products such as oranges, in particular those of the immature bitter oranges.

Thus, for example, oranges and their components may be considered part of a natural diet. This includes all parts of the orange, even those less commonly employed, as they may be eaten if wished given that oranges are generally considered to be non-toxic. Hence, bioflavonoids from different orange types and different parts of the oranges may be used to provide the mixtures of bioflavonoids described herein. A preferred source is the pith of the bitter orange, such as the immature bitter orange. This can be in the mixtures set out in Reference Examples 1 and 2 hereinafter.

The unit dosage of nutritional supplement for use as described herein will contain naringin and neohesperidin in the amounts as herein described, aptly together with further bioflavonoids. Such further bioflavonoids are aptly also derivable from the pith of the immature bitter oranges, and may be one or more of those set out in Reference Examples 1 and 2 herein.

Amounts of yet other bioflavonoids (for example, from other citrus fruits) may also be included, as long as they do not reduce the total content of naringin and neohesperidin to below 60% wt/wt of the total mixture of bioflavonoids present, preferably not below 75% wt/wt of the total mixture of bioflavonoids present.

The flavonoids mixture may also contain other non-flavonoid compounds of the extract from an edible plant source, for example from a further citrus. Similarly, the nutritional supplement may use carbohydrates from the fruit providing the flavonoids. Thus, the nutritional supplement may also comprise other biomass from the pith of the bitter orange. This enables the avoidance of complex extraction processes, which would be required if individual pure flavanones needed to be prepared and then blended, and can lead to a more readily solubilized mixture of flavonoids.

If desired, the compositions may also comprise naturally derivable acids such as citric acid, lactic acid, tartaric acid, malic acid, caprylic acid, ascorbic acid, acetic acid, and the like.

The nutritional supplement may also comprise a buffering agent, especially if provided in liquid form or in a dispersible or dissolvable solid form prior to ingestion, when a pH of 3-8, for example 4-7, is apt in the solution or dispersion which is then taken.

The nutritional supplement may also comprise agents to help solubilisation. Thus, for example, agents such as cyclodextrins (such as beta cyclodextrin or hydroxypropyl cyclodextrin) and/or a glycosaminoglycan (such as a hyaluronic acid) may be present.

Capsules may aptly be hard gelatine capsules into which the desired amount of nutritional supplement may be placed. These may be prepared in the conventional manner. The supplement components may be blended and, for example, granulated prior to placement in the capsule if desired.

Tablets may, if desired, be formulated for controlled or rapid dispersion in the stomach or in, for example, water or a beverage prior to ingestion. Such tablets may include conventional agents, leading to rapid dispersion or dissolution in accordance with standard pharmaceutical practice known to the skilled formulation expert. One form of favoured rapid dispersal tablet incorporates an effervescent base, such as citric acid (or other conventional acid such as tartaric, lactic or malic acid) and a carbonate or bicarbonate such as sodium or potassium bicarbonate. Such effervescent tablets may be formulated by conventional methods known to the skilled person.

Solid dosage forms, such as capsules and tablets, and especially effervescent tablets may be favoured when larger amounts of bioflavonoids are present in a unit dose, such as 200-2500 mg, for example about 250, 300, 350, 400, 450, 500, 600, 750, 1000, 1250, 1500 or 2000 mg. This is apt, as when employed together with other biomass from the fruit as described in Reference Example 2 herein, the tablets can be large and dissolution in water or a beverage is helpful prior to drinking. The beverage may be a still drink or a carbonated drink (sometimes referred to as a soda) as desired.

In an alternative, when particularly such larger amounts of bioflavonoids are employed, a chewable tablet, or one which disperses rapidly in saliva, may be employed.

The nutritional supplement may be taken with or between meals, although some subjects found use at mealtime more convenient.

Conditions for which the nutritional supplement is contemplated for use include those of inflammation of the gastrointestinal tract and those forming metabolic dysfunctions collectively referred to as insulin resistance syndrome or metabolic syndrome and even immune dysfunction originating in adverse gut conditions. The finding that in human subjects, such conditions are ameliorated by use of the nutritional supplements described herein suggests with the benefit of hindsight having read the Examples herein, and possibly due to an improvement in intestinal biota, which in turn improves gut wall health, which can then lead to better control of glucose spikes and gut irritation and permeability. While not wishing to be bound by such a theory, it is suggested to explain the diverse conditions which are ameliorated by use of the nutritional supplements herein as set out in the Examples.

The observation that central adiposity and triglyceride levels may be reduced when taking the nutritional supplement for the treatment of other conditions leads to the conclusion that use of the nutritional supplements herein is of benefit in the treatment of non-alcoholic fatty liver disease.

The observation that kidney function may be improved when taking the nutritional supplement for other conditions likewise indicates a role for the nutritional supplements herein for treating chronic kidney disease, especially mild disease (stage 3), for example as associated with poor sugar, as in diabetes type 2.

### Reference Example 1

A suitable supplement may contain bioflavonoids from bitter oranges, as follows:

| Bioflavonoid | % of Total Bioflavonoid Component |
|---|---|
| Isocriocirm | 2.4 |
| Isonaringin | 2.7 |
| Naringin | 52.0 |
| Hesperidine | 3.1 |
| Neohesperidin | 27.8 |
| Neodiomin | 3.1 |
| Naringenin | 3.4 |
| Poncirin | 4.4 |
| Rhiofolen | 1.1 |
| Total | 100% |

### Reference Example 2

A suitable supplement may contain bioflavonoids as set forth in Reference Example 1, together with other biomass derived from the pith of immature bitter oranges in the wt/wt ratio of 5:1 to 1:5, such as 2:1 to 1:2, and most aptly 1.25:1 to 1:1.25, and preferably 45:55. A preferred mixture is:

| Bioflavonoid | % in total biomass |
|---|---|
| Isocriocirm | 1.1 |
| Isonaringin | 1.2 |
| Naringin | 23.4 |
| Hesperidine | 1.4 |
| Neohesperidin | 12.5 |
| Neodiomin | 1.4 |
| Naringenin | 1.5 |
| Poncirin | 2.0 |
| Rhiofolen | 2.8 |

### Reference Example 3

### Liquid Formulation of Bioflavonoids

| | |
|---|---|
| Bioflavonoid of Ref. Ex. 2 above | 5% |
| Malic Acid | 15% |
| Citric Acid | 15% |
| Lactic Acid | 20% |
| Glycerine | 20% |
| Water | 25% |

### Reference Example 4

The tests were conducted on five groups of crossbred pigs of the Landrace Large White variety. Each group consisted of ten animals, which had been weaned at 21 days+/−3 days, and had a live body weight of 6 kg+/−0.5 kg.

The experimental groups were fed with diets constituted as follows:
Group “A”—Control feed
Group “B”—Standard commercial feed (control diet)
Group “C”—Control diet with the addition of 40 mg/kg feed of antibiotics
Group “D”—Control diet with the addition of 500 mg/kg feed of bioflavonoids of Reference Example 3
Group “E”—Control diet with the addition of 1000 mg/kg feed of bioflavonoids of Reference Example 3

To finalize the trials, three pigs from each group were slaughtered in order to permit a microbiological study of their intestinal tracts. This examination extended along the whole of the gastrointestinal tract from the stomach to the rectum, at intervals throughout their length. Sterile and refrigerated conditions were maintained in the laboratory throughout the analytical process.

The identity of the animals slaughtered in each group was as follows:
Group “A”—animals 1, 12 & 22
Group “B”—animals 19, 20 & 39
Group “C”—animals 13, 14 & 47
Group “D”—animals 26, 36 & 43
Group “E”—animals 4, 17 & 18

For each animal the microbiological studies were carried out in four zones, distributed along the digestive tract, and the results for these four zones in each of the five animal groups, are referred to as A1-A4, B1-B4, C1-C4, D1-D4 and E1-E4.

The microbiological analytical parameters used were:
1. Total count of all aerobic bacteria, expressed as UFC/g.
2. Total count of anaerobic bacteria, expressed as UFC/g.
3. Total count of filamentary fungi and moulds, expressed as UFC/g.
4. Total count of Enterobacteriaceae species, expressed as UFC/g.
5. Presence or absence of Salmonella species.
6. Total count of Lactobacillus species, expressed as UFC/g.

The methods used in the microbiological tests were those recommended by the Microbiological Clinic for Feeds.

The detailed results obtained have been summarised in Tables Numbers 1-5 on the following pages. They are expressed as the mean values for the three animals examined from each group.

TABLE 1
Results corresponding to the control feed group.

| Pathogen Type | A1 | A2 | A3 | A4 |
|---|---|---|---|---|
| Aerobic bacteria | 6 × 10⁸ | 5 × 10⁷ | 3 × 10⁷ | 8 × 10⁸ |
| Anaerobic bacteria | 7 × 10² | 6 × 10² | 4 × 10² | 6 × 10⁶ |
| Fungi | 2 × 10² | 2 × 10² | 3 × 10² | 4 × 10² |
| Enterobacteriaceae | 2 × 10³ | 3 × 10⁴ | 2 × 10⁵ | 7 × 10⁵ |
| Salmonella | Present | Present | Present | Present |
| Lactobacillus | 4 × 10⁴ | 3 × 10⁴ | 2 × 10⁴ | 2 × 10⁴ |

TABLE 2
Results corresponding to the commercial feed group.

| Pathogen Type | B1 | B2 | B3 | B4 |
|---|---|---|---|---|
| Aerobic bacteria | 3 × 10⁸ | 3 × 10⁷ | 2 × 10⁵ | 5 × 10⁸ |
| Anaerobic bacteria | 4 × 10⁶ | 5 × 10⁶ | 4 × 10⁶ | 5 × 10⁶ |
| Fungi | 3 × 10² | 2 × 10³ | 2 × 10⁴ | 2 × 10⁴ |
| Enterobacteriaceae | 2 × 10³ | 3 × 10³ | 4 × 10⁵ | 5 × 10⁵ |
| Salmonella | Present | Present | Present | Present |
| Lactobacillus | 6 × 10⁴ | 3 × 10⁴ | 2 × 10⁴ | 2 × 10⁴ |

TABLE 3
Results corresponding to the control feed with the
addition of 40 mg of antibiotics per kg of feed.

| Pathogen Type | C1 | C2 | C3 | C4 |
|---|---|---|---|---|
| Aerobic bacteria | 8 × 10⁶ | 4 × 10⁶ | 2 × 10⁶ | 2 × 10⁶ |
| Anaerobic bacteria | 6 × 10⁷ | 6 × 10⁷ | 2 × 10⁷ | 3 × 10⁴ |
| Fungi | 4 × 10² | 4 × 10³ | 4 × 10³ | 3 × 10⁴ |
| Enterobacteriaceae | 6 × 10² | 5 × 10² | 5 × 10⁴ | 5 × 10⁴ |
| Salmonella | Absent | Absent | Absent | Absent |
| Lactobacillus | 6 × 10³ | 4 × 10³ | 5 × 10³ | 2 × 10³ |

TABLE 4
Results corresponding to the control feed with the addition of
500 mg of bioflavonoids of Reference Example 3 per kg of feed.

| Pathogen Type | D1 | D2 | D3 | D4 |
|---|---|---|---|---|
| Aerobic bacteria | 6 × 10⁵ | 6 × 10⁵ | 6 × 10⁶ | 4 × 10⁶ |
| Anaerobic bacteria | 2 × 10³ | 4 × 10³ | 4 × 10³ | 4 × 10³ |
| Fungi | 3 × 10² | 2 × 10⁴ | 3 × 10⁴ | 3 × 10⁴ |
| Enterobacteriaceae | 2 × 10² | 3 × 10² | 6 × 10³ | 3 × 10³ |
| Salmonella | Absent | Absent | Absent | Absent |
| Lactobacillus | 7 × 10⁴ | 5 × 10⁴ | 6 × 10⁴ | 2 × 10⁴ |

TABLE 5
Results corresponding to the control feed with the addition of
1000 mg of bioflavonoids of Reference Example 3 per kg of feed.

| Pathogen Type | E1 | E2 | E3 | E4 |
|---|---|---|---|---|
| Aerobic bacteria | 3 × 10⁵ | 3 × 10⁵ | 4 × 10⁶ | 4 × 10⁶ |
| Anaerobic bacteria | 2 × 10³ | 4 × 10³ | 4 × 10³ | 4 × 10³ |
| Fungi | 2 × 10³ | 2 × 10⁴ | 1 × 10⁴ | 2 × 10⁴ |
| Enterobacteriaceae | 2 × 10² | 3 × 10² | 2 × 10³ | 3 × 10³ |
| Lactobacillus | 7 × 10⁴ | 5 × 10⁴ | 6 × 10⁴ | 2 × 10⁴ |

The presence of Salmonella species in the animals fed with the control diet and the commercial diet does not indicate that the feed was in poor condition, since Salmonella form a part of the natural microbial content of the animals, with no clinical implications.

The fact of the presence of Lactobacillus species in animals fed with both the treated feed, and the antibiotic treated feed, was a favourable factor for the growth of the animals.

The variations in the counts of the various groups of microorganisms were explained by the method of administration of the substances, which diminishes the total aerobic bacterial content compared with the counts for the anaerobic bacteria.

In those examples where major concentrations of fungi were detected (by comparison with the control), there was a marked presence of moulds, basically of the genus Saccharomyces, resulting in a reduction of the pH level. This is favourable to the formation of conditions similar to those found in the presence of the Lactobacillus species.

The dosage level of 500 mg of bioflavonoids of Reference Example 3 per kilo of feed weight was sufficient to give excellent results in all cases. Only in those cases where there was evidence of a very high level of contamination was it necessary to increase the dosage to 1000 mg per kilo of feed.

The reduction in general of the bacteria present in the intestinal tract following the use of bioflavonoids was considered to be satisfactory and comparable with the reduction obtainable with antibiotics, to which they form a valid alternative.

Specifically, similar reductions were found in the quantities of Enterobacteriaceae and Salmonella species present.

### Reference Example 5

Administration of the nutritional supplement described herein (and in particular, that set forth in Reference Example 3 hereinafter) enhances the presence of short-chain fatty acids in the gut. This is generally accepted to correlate with good gut health (for example, a lower incidence of colon cancer). This increase in acetic, propionic and butyric acids may result from modification of the microbiome to increase the proportion of beneficial short-chain fatty acid-producing bacteria, such as Lactobacillus and Bifidobacteria, observed in stool samples.

### Example 1—Use of Nutritional Supplement in Ameliorating Type 2 Diabetes

A 59-year-old male subject with type 2 diabetes was treated with metformin (20×2 mg/day), which resulted in a stable condition. When treated for six months with 5 ml dissolved in 200 ml of water twice per day of bioflavonoid concentrate as described in Reference Example 3, the subject's glycosylated haemoglobin decreased from approximately 8 at the start of supplementation to approximately 6 at the end of the period of supplementation with the nutritional mixture of flavonoids.

Without changing diet or exercise, the subject's weight declined by 7 kg and waist measurement decreased by 5 cm, indicating a loss of central adiposity.

### Example 2—Use of Nutritional Supplement in Ameliorating Prediabetes

A group of 4 males having glycosylated haemoglobin in the prediabetes range had their diet supplemented each day with 5 g of the bioflavonoids of Reference Example 3 dissolved in 200 ml of water. Over a six-month period, the average glycosylated haemoglobin declined from about 7 to about 6, indicating a reversal of progression to type 2 diabetes.

### Example 3—Use of Nutritional Supplement in Amelioration of Duodenal Ulcers

A male subject suffering from a duodenal ulcer (due to H. pylori) treated with a proton pump inhibitor supplemented his diet with 5 g of the bioflavonoids of Reference Example 3 dissolved in 200 ml of water twice each day for six months. By the end of this time, subjective pain score had reduced significantly. Medical advice was received to the effect that regression of the ulcer had occurred. The subject was able to cease the use of omeprazole. The H. pylori infection abated.

### Example 4—Use of Nutritional Supplement to Normalize Bowel Movements

After one month's treatment with 15 ml three times per day of the bioflavonoids of Reference Example 3 as a mouthwash (diluted to about 300 ml with effervescent cola), stool samples of a subject changed from Bristol score 1 to Bristol score 4, accompanied by a reduction in constipation and development of normal bowel movements.

### Example 5—Use of Nutritional Supplement to Normalize Blood Lipids

A 70+-year-old subject suffering from very high triglyceride levels (14 mmol/l, normal reference range maximum 1.7 mmol/1) was placed on a diet and exercise regimen by an endocrinologist. This produced a substantial improvement in triglycerides (reduction to 2.0 mmol/1) after 1 year. The subject subsequently self-administered bioflavonoids of Reference Example 3 mouthwash (15 ml, 3 times a day, diluted to about 300 ml with effervescent cola) with meals for three months. Triglycerides reduced to normal (1.4 mmol/1). Nutritional supplementation continued and a subsequent blood test showed reduction in low-density cholesterol to within the normal range and an increase in high-density cholesterol into the normal range. The endocrinologist discharged the patient as no longer requiring care. By the end of this time, blood pressure was 125/75 mm/Hg (reduced from 150/80) and kidney function improved from stage 3 renal failure to stage 2 renal failure. The subject's previously stable weight declined by 2 kg and waist measurement by 2 cm during the period of administration of the nutritional supplement.

### Example 6—Capsule Formulation

A hard gelatine capsule dose was employed. Into this was placed 2 g of the mixture of Reference Example 2 to provide a unit dosage containing approximately 1 g of mixed bioflavonoids.

The capsule may be taken, for example, 1, 2, or 3 times a day to supplement the diet in order to treat conditions referred to herein, such as those referred to in Examples 1-5.

An analogous but smaller capsule form was prepared containing approximately 250-500 mg of bioflavonoids by hard filling capsules with 1 g or 500 mg of Reference Example 2 and hand closing the capsules.

### Example 7—Tablet Formulation

An effervescent tablet formulation may be prepared on standard tabletting equipment using the following blended components:
Bioflavonoids 2 g of Reference Example 2
Tartaric acid 2 g
Sodium bicarbonate 2 g
Sweetener (Stevia) 0.1 g

The sweetener (Stevia) was employed to render the solution obtained by dissolution of one or two tablets in water (200 ml) more palatable, as otherwise the bitter taste of the natural orange nutritional supplement is apparent. The sweetener employed is chosen to be naturally occurring so as to enhance the plant-derived nutritional supplement nature of the tablets, which can be used to provide solutions to be drunk to ameliorate the conditions referred to herein, such as those referred to in of Examples 1-5.

### Example 8—Sachet Formulations

The mixture of Reference Example 2, 2.5 g, may be granulated with microcrystalline cellulose and lactose and sealed into a sachet in a conventional manner. The granules dissolve when added to water to provide a solution which can be drunk to ameliorate conditions referred to herein.

1. A nutritional supplement for use by oral administration to a human for ameliorating adverse medical conditions deriving at least in part from a metabolic dysfunction and/or an intestinal dysfunction, which supplement is in a form suitable for oral administration and comprises a mixture of bioflavonoids which include at least 45% wt/wt of naringin and at least 15% wt/wt of neohesperidin of the bioflavonoids present.

2. A method of ameliorating an adverse medical condition in a human which derives at least in part from a metabolic and/or intestinal dysfunction which method comprises the oral administration of an effective amount of a nutritional supplement which comprises a mixture of bioflavonoids which include at least 45% wt/wt of naringin and at least 15% wt/wt of neohesperidin of the bioflavonoids present.

3. The supplement of claim 1 wherein the supplement comprises at least 50% of naringin and at least 20% of neohesperidin of the bioflavonoids present.

4. The supplement of claim 3, wherein the supplement comprises at least 75% wt/wt, for example 80-90% wt/wt, of naringin and neohesperidin of the bioflavonoids present.

5. The supplement claim 1, wherein the mixture of bioflavonoids is derived from the pith of immature bitter oranges.

6. The supplement of claim 1, wherein the adverse medical condition is selected from the group comprising (a) an insulin resistance syndrome condition, wherein said insulin resistance syndrome condition is selected from the group consisting of type 2 diabetes, prediabetes and dyslipidaemia; (b) a gastrointestinal ulcer, irritable bowel syndrome or leaky gut syndrome; (c) a duodenal ulcer; (d) central adiposity; and (e) hypertension.

7.-10. (canceled)

11. The supplement of claim 1, wherein the oral administration comprises 100 mg-5 g/day, more aptly 250 mg-2 g/day, for example 0.5-1.5 g/day of the mixture of bioflavonoids, such as 1 g/day.

12. The supplement of claim 11, wherein the supplement is in the form of a multidose liquid from which a unit dose may be taken.

13. The supplement of claim 11, wherein the supplement is in the form of a solid unit dose.

14. The supplement of claim 13, wherein the solid unit dose is in a form selected from the group consisting of (a) a capsule form, (b) a hard gelatine capsule, (c) a water-dispersible or soluble tablet, (d) a granular form within a sachet, (e) an effervescent tablet, (f) water-dispersible or soluble granules and (g) effervescent granules.

15.-17. (canceled)

18. The supplement of claim 1, wherein the nutritional supplement also comprises biomass derived from the pith or immature bitter oranges.

19.-22. (canceled)

23. The claim 2 wherein the supplement is administered from 1-6 times a day, most suitably 2-4 times a day, to provide a daily dose of 0.5-2 g of the mixture of bioflavonoids per day.

24. The method of claim 23, wherein the supplement is administered on 2, 3 or 4 occasions per day to provide 0.75-1.5 g per day of the mixture of bioflavonoids.

25. An orally administrable solid unit dose formulation containing a nutritional supplement as set forth in claim 1 for use in ameliorating a gastrointestinal or metabolic condition.

26. AP The orally administered solid unit dose formulation of claim 25, wherein the gastrointestinal or metabolic condition is selected from the group consisting of type 2 diabetes, a gastric ulcer, a duodenal ulcer, constipation, elevated serum lipid levels, elevated serum triglyceride levels, adiposity, central adiposity, non-alcoholic fatty liver disease, and leaky gut syndrome.

27.-33. (canceled)

34. The solid unit dose formulation claim 25, in the form of a capsule.

35. The capsule of claim 25, which contains 100 mg-2 g of the mixture of bioflavonoids, for example 250-1200 mg of the mixture of bioflavonoids, such as 250-1000 mg of the mixture of bioflavonoids.

36. The solid unit dose formulation of claim 25, in the form of a tablet, such as a dispersible, effervescent or chewable tablet, containing 100 mg-2 g of the mixture of bioflavonoids.
